(19) United States
(12) Patent Application Publication
(10) Pub. No.: US 2026/0250314 A1
(43) Pub. Date: Aug. 27, 2026

(54) POTATO PROTEIN EXTRACTION APPARATUS AND METHOD

(71) Applicant: Root Extracts Limited, Lincoln (GB)

(72) Inventors: Vidyanath Gururajan, Lincoln (GB); Alison Wright, Lincoln (GB)

(21) Appl. No.: 18/878,242

(22) PCT Filed: Jun. 21, 2023
(86) PCT No.: PCT/GB2023/051627

§ 371 Date: Dec. 23, 2024

(30) Foreign Application Priority Data
Jun. 23, 2022 (GB) 2209211.8

(51) Int. Cl. C07K 1/34; A23J 1/00; B01D 21/26; B01D 29/27; B01D 29/80; B01D 36/02; B01D 36/04; B01D 37/00; B01D 61/14; B01D 61/16; B01D 61/18; B01D 69/02; B04B 3/00; C07K 14/415

(52) U.S. Cl. CPC C07K 1/34; A23J 1/006; B01D 21/262; B01D 29/27; B01D 29/80; B01D 36/02; B01D 36/045; B01D 37/00; B01D 61/145; B01D 61/16; B01D 61/18; B01D 69/02; B04B 3/00; C07K 14/415; B01D 2201/184; B01D 2311/04; B01D 2315/16; B01D 2325/20

(57) ABSTRACT

Apparatus for extracting protein from potatoes, the apparatus includes a first filtration device having a filter material with a first pore size; a second filtration device having a filter material with a second pore size; a third filtration device having a filter material with a third pore size; and an ultrafiltration device, the second pore size is smaller than the first pore size, and the third pore size is smaller than the second pore size.

### CROSS-REFERENCE TO RELATED APPLICATIONS

This application claims priority to International Application No. PCT/GB2023/051627, filed on Jun. 21, 2023, entitled “POTATO PROTEIN EXTRACTION APPARATUS AND METHOD,” which claims the benefit of United Kingdom Patent Application GB2209211.8, filed on Jun. 23, 2022, entitled “POTATO PROTEIN EXTRACTION APPARATUS AND METHOD,” the disclosures of which are all hereby incorporated herein by reference in their entirety.

### FIELD OF THE DISCLOSURE

The present disclosure relates to apparatuses and methods for potato protein extraction. More specifically, the disclosure relates to apparatuses and methods for potato protein extraction where the protein is extracted by filtration through multiple filtration devices.

### BACKGROUND

Proteins are an important part of everyday dietary requirement. Proteins are required for various crucial biological processes including muscle synthesis, DNA replication, catalysing metabolic reactions, etc. Lack of protein intake leads to serious conditions such as muscle atrophy and liver failure. In recent years, vegetarianism has been on the rise due to reasons such as animal welfare, environmental issues associated with farming, lactose intolerance and weight management. Therefore, there is a thriving market for providing plant-based proteins. Potatoes contain proteins that have a nutritional value able to substitute animal proteins, such as those derived from meat, eggs, milk, etc.

Potato proteins are usually obtained from potato fruit juice. It is known to use filtration methods to extract the protein from the potato fruit juice. A disadvantage associated with such known methods is that the filtration devices become fouled by particulates from the potato fruit juice, thereby causing limits in production efficiency. Filters need to be cleaned or replaced which can result in process downtime and/or increased costs.

It is therefore a non-exclusive object to overcome or at least substantially alleviate one or more problems associated with the prior art.

Embodiments of the present invention seek to alleviate one or more problems associated with the prior art.

### BRIEF SUMMARY

According to a first aspect, there is provided an apparatus for extracting protein from potatoes, the apparatus comprising: a first filtration device having a filter material with a first pore size; a second filtration device having a filter material with a second pore size; a third filtration device having a filter material with a third pore size; and an ultrafiltration device, wherein the second pore size is smaller than the first pore size, and the third pore size is smaller than the second pore size.

The first filtration device, the second filtration device and/or the third filtration device may be integrated into a single unit. Alternatively, the first filtration device, the second filtration device and/or the third filtration device may be discrete (standalone) units.

The apparatus may further comprise a decanter centrifuge and/or a sedimentation vessel for clarifying potato fruit juice. Clarification is a process of separating insoluble particulates from liquid. In the methods described herein, the potato fruit juice may be clarified to obtain clarified potato fruit juice.

The first pore size may be from 10 micron to 200 micron, such as from 10 micron to 100 micron, such as from 10 micron to 50 micron, such as from 25 micron to 50 micron. For example, the first pore size may be 10 micron, 15 micron, 20 micron, 25 micron, 30 micron, 35 micron, 40 micron, 45 micron, 50 micron, 55 micron, 60 micron, 65 micron, 70 micron, 75 micron, 80 micron, 85 micron, 90 micron, 95 micron, 100 micron, 105 micron, 110 micron, 115 micron, 120 micron, 125 micron, 130 micron, 135 micron, 140 micron, 145 micron, 150 micron, 155 micron, 160 micron, 165 micron, 170 micron, 175 micron, 180 micron, 185 micron, 190 micron, 195 micron or 200 micron.

The second pore size may be from 0.8 micron to 10 micron, such as from 0.8 micron to 5micron, such as from 0.8 micron to 4 micron, such as from 0.8 micron to 3 micron, such as from 0.8 micron to 2 micron. For example, the second pore size may be 0.8 micron, 1.0 micron, 1.2 micron, 1.4 micron, 1.6 micron, 1.8 micron, 2.0 micron, 2.2 micron, 2.4 micron, 2.6 micron, 2.8 micron, 3.0 micron, 3.2 micron, 3.4 micron, 3.6 micron, 3.8 micron, 4.0 micron, 4.2 micron, 4.4 micron, 4.6 micron, 4.8 micron, 5.0 micron, 5.2 micron, 5.4 micron, 5.6 micron, 5.8 micron, 6.0 micron, 6.2 micron, 6.4 micron, 6.6 micron, 6.8 micron, 7.0 micron, 7.2 micron, 7.4 micron, 7.6 micron, 7.8 micron, 8.0 micron, 8.2 micron, 8.4 micron, 8.6 micron, 8.8 micron, 9.0 micron, 9.2 micron, 9.4 micron, 9.6 micron, 9.8 micron or 10 micron.

The third pore size may be less than 0.8 micron, such as from 0.1 micron to less than 0.8 micron, such as from 0.2 micron to 0.7 micron, such as from 0.3 micron to 0.6 micron, such as from 0.4 micron to 0.5 micron. For example, the third pore size may be 0.1 micron, 0.2 micron, 0.3 micron, 0.4 micron, 0.5 micron, 0.6 micron or 0.7 micron.

The ultrafiltration device may comprise a tangential flow ultrafiltration device.

The ultrafiltration device may comprise one or more filter membrane(s) having a molecular weight cut-off of from 5 kDa to 150 kDa, such as from 5 kDa to 100 kDa, such as from 5 kDa to 50 kDa, such as from 5 kDa to 25 kDa, such as from 5 kDa to 10 kDa. For example, the molecular weight cut-off may be 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 105 kDa, 110 kDa, 115 kDa, 120 kDa, 125 kDa, 130 kDa, 135 kDa, 140 kDa, 145 kDa or 150 kDa.

The or each filter membrane may have a membrane surface area of 3.5 m2 to 40 m2. For example, each filter membrane may have a membrane surface area of 4.1 m2. The or each filter membrane may be made from polyethersulfone on a polyester backing material. The or each filter membrane may have a maximum operating temperature of 50-85° C. The or each filter membrane may have a maximum operating pressure of 970 kPa. The or each filter membrane may be a spiral membrane filter. In some examples, the ultrafiltration device may comprise multiple (e.g. two) spiral membrane filters.

The filter material of any one or more of the first filtration device, the second filtration device and the third filtration device may be in the form of a bag filter.

The bag filter or bag filters may be able to withstand temperatures up to 150° C., such as up to 95° C. The bag filter or bag filters may be able to withstand pressure differentials of up to approximately 100 kPa. The bag filter or bag filters may have a substantially circular opening having a diameter, for example, of approximately 0.1 m to 0.2 m. The diameter may be 0.102 m or 0.178 m, for example. The bag filter may have a length of approximately 0.2 m to 1 m. The length may be 0.229 m, 0.356 m, 0.406 m or 0.813 m, for example. The total area of the filter material of the bag filter or bag filters may be from 0.05 m2 to 0.5 m2. The total area may be 0.07 m2, 0.12 m2, 0.23 m2 or 0.41 m2, for example. In one example, the diameter of the bag filter may be 0.178 m, the length may be 0.406 m and the total area may be 0.23 m2.

Any one or more of the first filtration device, the second filtration device and the third filtration device may comprise or consist of a centrifugal separator comprising: a housing having: an inlet for receiving potato fruit juice; a first outlet; and a second outlet; an element configured to rotate relative to the housing to create centrifugal forces within the housing; and the filter material of the respective first filtration device, second filtration device and/or third filtration device, wherein the first outlet is configured to receive retentate that has been retained by the filter material and the second outlet is configured to receive filtrate that has passed through the filter material.

In some examples, the filter material may be in the form of a woven or non-woven fabric, e.g., felt. In some examples, the filter material may be of a mesh, net-like or porous construction. The filter material may be made from polypropylene, polyester, nylon, porous polytetrefluoroethylene (PTFE) film, fluoropolymers, viscose, aromatic polyamides, wool, stainless steel, or a combination thereof. The skilled person would readily appreciate that other materials may be used to form the filter material-this list of materials is non-exhaustive.

The filter material of any one or more of the first filtration device, the second filtration device and the third filtration device may comprise one or more layers, e.g., up to ten layers. For example, the filter material of any filtration device may comprise one, two, three, four, five, six, seven, eight, nine or ten layers.

The filter material of the first filtration device and/or the filter material of the second filtration device may comprise one layer.

The filter material of the third filtration device may comprise two or more layers.

When the filter material of any one of the first filtration device, the second filtration device and the third filtration device comprises two or more layers, the layers may have differing pore sizes. For example, the layers may have diminishing pore sizes going from a first side of the filter material to a second side of the filter material opposite to the first side. In such examples, the pore size of the filter material is equivalent to that of the layer with the smallest pore size.

The apparatus may further comprise a drying device for drying potato protein. The drying device may comprise any one or a combination of a spray dryer, freeze dryer or an atmospheric belt dryer. In examples comprising a spray dryer, the spray dryer may include a nozzle operable to spray fluid into a hot air stream. The hot air stream may have a temperature range of from 120° C. to 600° C. In examples comprising a freeze dryer, the freeze dryer may have a temperature range from −50° C. to −80° C. As will be appreciated by the skilled person, alternative drying methods may be used.

According to a second aspect, there is provided a method for extracting protein from potatoes, the method comprising: a) obtaining potato fruit juice; b) passing the potato fruit juice through a first filtration device having a filter material with a first pore size to obtain a first filtrate; c) passing the first filtrate through a second filtration device having a filter material with a second pore size to obtain a second filtrate; d) passing the second filtrate through a third filtration device having a filter material with a third pore size to obtain a third filtrate; and e) ultrafiltering the third filtrate to obtain an ultrafiltration retentate, wherein the second pore size is smaller than the first pore size, and the third pore size is smaller than the second pore size.

The method may further comprise drying the ultrafiltration retentate to produce the potato protein.

The drying step may result in a powder having greater than 65% by weight native potato protein, such as 70% by weight native potato protein, such as greater than 75% by weight native potato protein, such as greater than 80% by weight native potato protein, such as greater than 85% by weight native potato protein, such as greater than 90% by weight native potato protein, such as greater than 95% by weight native potato protein.

The first pore size may be from 10 to 200 micron, such as from 10 micron to 100 micron, such as from 10 micron to 50 micron, such as from 25 micron to 50 micron. For example, the first pore size may be 10 micron, 15 micron, 20 micron, 25 micron, 30 micron, 35 micron, 40 micron, 45 micron, 50 micron, 55 micron, 60 micron, 65 micron, 70 micron, 75 micron, 80 micron, 85 micron, 90 micron, 95 micron, 100 micron, 105 micron, 110 micron, 115 micron, 120 micron, 125 micron, 130 micron, 135 micron, 140 micron, 145 micron, 150 micron, 155 micron, 160 micron, 165 micron, 170 micron, 175 micron, 180 micron, 185 micron, 190 micron, 195 micron or 200 micron.

The second pore size may be from 0.8 micron to 10 micron, such as from 0.8 micron to 5 micron, such as from 0.8 micron to 4 micron, such as from 0.8 micron to 3 micron, such as from 0.8 micron to 2 micron. For example, the second pore size may be 0.8 micron, 1.0 micron, 1.2 micron, 1.4 micron, 1.6 micron, 1.8 micron, 2.0 micron, 2.2 micron, 2.4 micron, 2.6 micron, 2.8 micron, 3.0 micron, 3.2 micron, 3.4 micron, 3.6 micron, 3.8 micron, 4.0 micron, 4.2 micron, 4.4 micron, 4.6 micron, 4.8 micron, 5.0 micron, 5.2 micron, 5.4 micron, 5.6 micron, 5.8 micron, 6.0 micron, 6.2 micron, 6.4 micron, 6.6 micron, 6.8 micron, 7.0 micron, 7.2 micron, 7.4 micron, 7.6 micron, 7.8 micron, 8.0 micron, 8.2 micron, 8.4 micron, 8.6 micron, 8.8 micron, 9.0 micron, 9.2 micron, 9.4 micron, 9.6 micron, 9.8 micron or 10 micron.

The third pore size may be less than 0.8 micron, such as from 0.1 micron to less than 0.8 micron, such as from 0.2 micron to 0.7 micron, such as from 0.3 micron to 0.6 micron, such as from 0.4 micron to 0.5 micron. For example, the third pore size may be 0.1 micron, 0.2 micron, 0.3 micron, 0.4 micron, 0.5 micron, 0.6 micron or 0.7 micron.

The potato fruit juice may be clarified prior to passing through the first filtration device.

The potato fruit juice may be clarified by any one or both of decantation and sedimentation techniques that are well known to the skilled person.

Step (e) of the method may involve subjecting the ultrafiltration retentate to diafiltration using well known techniques.

### BRIEF DESCRIPTION OF THE DRAWINGS

Examples of the disclosure will now be described with reference to the accompanying drawings, in which:

FIG. 1 is a schematic showing an apparatus for extracting protein according to a first example;

FIG. 2 is a schematic showing an apparatus for extracting protein according to a second example;

FIG. 3 is a schematic cross-sectional side view of a centrifugal separator; and

FIG. 4 is a schematic exploded view of a filter material comprising multiple layers.

### DETAILED DESCRIPTION

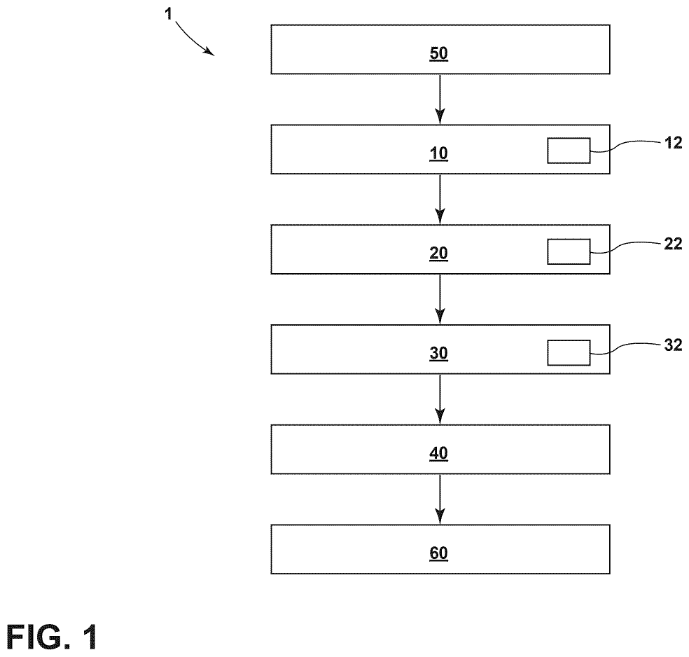

Referring to FIG. 1, there is shown an apparatus 1 for extracting protein according to a first example of the disclosure. The apparatus 1 comprises a first filtration device 10 having a filter material 12 with a first pore size, a second filtration device 20 having a filter material 22 with a second pore size and a third filtration device 30 having a filter material 32 with a third pore size.

Any one of the first filtration device 10, the second filtration device 20 and the third filtration device 30 may comprise or consist of a centrifugal separator 14. In particular, the first filtration device 10 may be a centrifugal separator 14.

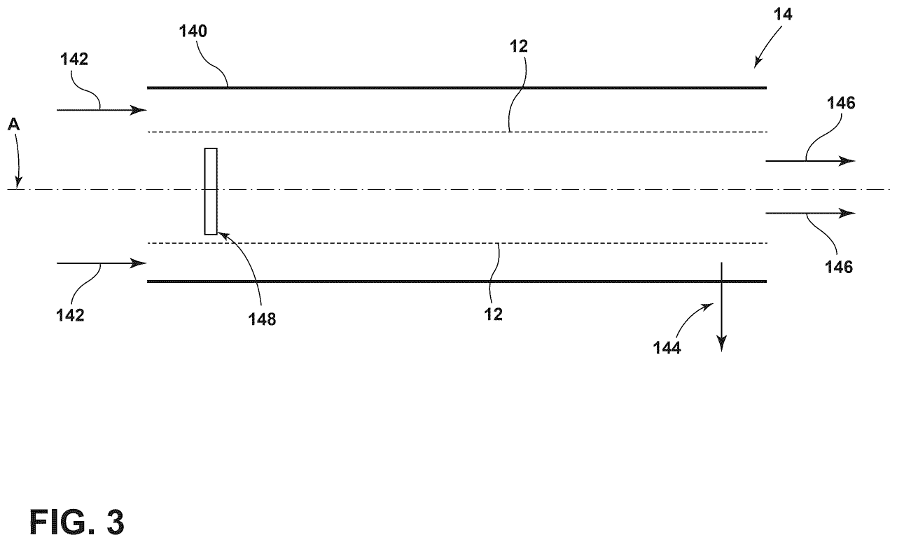

Referring to FIG. 3, the centrifugal separator 14 may comprise a housing 140, an element 148 that is configured to rotate relative to the housing 140 to create centrifugal forces within the housing 140, and the filter material 12 (in examples where the first filtration device 10 is the centrifugal separator 14). The housing 140 may have a substantially cylindrical shape having a central axis A. The filter material 12 may be positioned concentrically within the housing 140. The filter material 12 may be a wire mesh formed from, for example, stainless steel. The filter material 12 may have a pore size of 25 micron or 50 micron. The housing 140 may have an inlet 142 for receiving potato fruit juice, a first outlet 144 and a second outlet 146. During use, the element 148 may be configured to rotate about the axis A to create a vortex within the housing 140. Filtrate (i.e., liquids and particles that can pass through the filter material 12) is drawn along the vortex and exits the housing 140 at the second outlet 146. Retentate (i.e., particles that are too large to pass through the filter material 12) exits the housing at the first outlet 144. The housing 140 may have a capacity of 1.6 m3. An example centrifugal separator 14 may be of the type manufactured by Russell Finex.

Referring back to FIG. 1, the filter material 22, 32 of the respective second filtration device 20 and third filtration device 30 may be in the form of bag filters.

The filter material 22 (bag filter) of the second filtration device 20 may be made from polypropylene felt having a pore size of 1 micron. The filter material 22 may have a single layer. An example bag filter may be of the type manufactured by Fileder Filter Systems having part number of EBSP-1-1.

The filter material 32 (bag filter) of the third filtration device 30 may be made from polypropylene felt having a pore size of 0.5 micron. The filter material 32 may have five layers. An example bag filter may be of the type manufactured by Fileder Filter Systems having part number of PBP-0.5-1.

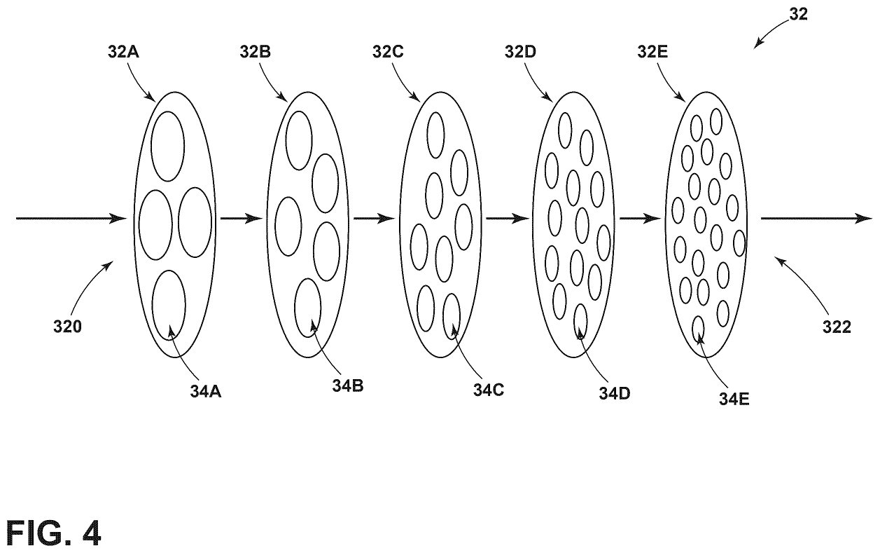

Referring now to FIG. 4, there is shown an exploded view of the filter material 32. The filter material has a first side 320 and a second side 322 opposite to the first side 320. The filter material 32 may comprise five layers 32A-E having differing pore sizes 34A-E. The layers 32A-E may have a diminishing pore size going from the first side 320 to the second side 322. The pore size of the filter material 32 (i.e., 0.5 micron) is equivalent to that of the layer 34E with the smallest pore size.

Referring back to FIG. 1, the apparatus 1 may further comprise a vessel or vessels 50 for clarifying potato fruit juice. The vessel or vessels 50 may include any one or both of a decanter centrifuge or a sedimentation vessel.

The apparatus 1 may further comprise an ultrafiltration device 40. The ultrafiltration device may comprise two or more spiral membrane filters having a molecular weight cut-off of 10 kDa.

The apparatus 1 may further comprise a drying device 60 in the form of a spray dryer.

A potato protein extraction method using the apparatus 1 will now be described. 1,500 kilograms of potatoes are sanitised and loaded into a rasper (not shown) where the potatoes are broken down into potato pulp. Water is added to the potato pulp to form potato fruit juice. The volume ratio of water to potato pulp may be from 1:2 to 1:1. For example, 1,000 litres of water may be mixed with 1,000 litres of potato pulp to form 2,000 litres of potato fruit juice. A preservative such as sodium metabisulphite may also be added. Alternatively, potato fruit juice may be obtained from another source. For example, potato fruit juice may be obtained directly from potato chip production facilities and the like.

The potato fruit juice is then fed into the vessel or vessels 50 to obtain clarified potato fruit juice. Alternatively, clarified potato fruit juice may be obtained from another source.

The clarified potato fruit juice is then fed into the first filtration device 10 which may be in the form of the centrifugal separator 14. The centrifugal separator 14 separates particles having a particle size of above 50 micron. Referring to FIG. 3, the clarified potato fruit juice is fed into the housing 140 via the inlet 142. The element 148 located inside the centrifugal separator 14 rotates the clarified potato fruit juice relative to the housing 140 such that centrifugal forces create a vortex. The filter membrane 12 prevents particles having a particle size of above 50 micron to pass therethrough. The retentate exits the housing at the first outlet 144. The remainder is drawn along the vortex and exits the housing 140 at the second outlet 146 to obtain a first filtrate.

The first filtrate is then fed into the second filtration device 20 which may be in the form of a bag filter. The second filtration device 20 has a filter material 22 with the pore size of 1 micron. The first filtrate enters the second filtration device 20 at a pressure of around 230 kPa. The first filtrate passes through the filter material 22 of the second filtration device 20, thereby removing particles having a particle size of above 1 micron to obtain a second filtrate.

The second filtrate is then fed into the third filtration device 30 which may be in the form of a bag filter. The filter material 32 of the bag filter may comprise five layers and may have a minimum pore size of 0.5 micron. The second filtrate enters the third filtration device 30 at a pressure of around 230 kPa. Referring to FIG. 5, the second filtrate flows in the direction of the arrows from the first side 320 to the second side 322. As the second filtrate flows through layers 32A-E, each layer 32A-E retains particles having a particle size larger than its respective pore size 34A-E, such that a third filtrate is obtained.

The third filtrate is then fed into the ultrafiltration device 40 to obtain an ultrafiltration retentate. Optionally, diafiltration using pure water or a buffer solution can be performed during ultrafiltration in accordance with known methods.

The ultrafiltration retentate is then fed into the drying device 60 which may be in the form of a spray dryer. The ultrafiltration retentate is sprayed into a stream of hot air to obtain a powder having greater than 70 % by weight native potato protein.

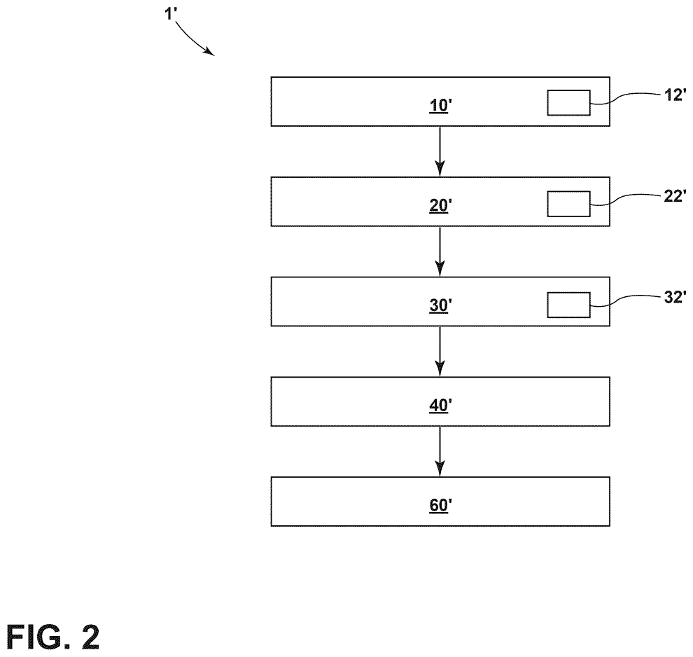

Referring to FIG. 2, there is shown an apparatus 1′ for extracting protein according to a second example, wherein parts of the apparatus 1′ similar to those of the first example are given the same number but are denoted with a prime (′).

The apparatus 1′ comprises a first filtration device 10′ having a filter material 12′ with a first pore size, a second filtration device 20′ having a filter material 22′ with a second pore size and a third filtration device 30′ having a filter material 32′ with a third pore size.

The filter material 12′, 22′, 32′ of the respective first filtration device 10′, second filtration device 20′ and third filtration device 30′ may be in the form of bag filters.

The filter material 12′ (bag filter) of the first filtration device 10′ may be made from polypropylene felt having a pore size of 50 micron. The filter material 12′ may have a single layer. An example bag filter may be of the type manufactured by Fileder Filter Systems having part number of EBSP-50-1.

The filter material 22′ (bag filter) of the second filtration device 20′ may be made from polypropylene felt having a pore size of 1 micron. The filter material 22′ may have a single layer. An example bag filter may be of the type manufactured by Fileder Filter Systems having part number of EBSP-1-1.

The filter material 32′ (bag filter) of the third filtration device 30′ may be made from polypropylene felt having a pore size of 0.5 micron. The filter material 32′ may have five layers. An example bag filter may be of the type manufactured by Fileder Filter Systems having part number of PBP-0.5-1.

The apparatus 1′ may further comprise an ultrafiltration device 40′. The ultrafiltration device may comprise two or more spiral membrane filters having a molecular weight cut-off of 10 kDa.

The apparatus 1′ may further comprise a drying device 60′ in the form of a spray dryer.

A potato protein extraction method using the apparatus 1′ will now be described. Clarified potato fruit juice is obtained and fed into the first filtration device 10′. The potato fruit juice enters the first filtration device 10′ at a pressure of around 230 kPa. The clarified potato fruit juice passes through the filter material 12′ of the first filtration device 10′, thereby removing particles having a particle size of above 50 micron to obtain a first filtrate.

The first filtrate is then fed into the second filtration device 20′ which may be in the form of a bag filter. The second filtration device 20′ has a filter material 22′ with the pore size of 1 micron. The first filtrate enters the second filtration device 20′ at a pressure of around 230 kPa. The first filtrate passes through the filter material 22′ of the second filtration device 20′, thereby removing particles having a particle size of above 1 micron to obtain a second filtrate.

The second filtrate is then fed into the third filtration device 30′ which may be in the form of a bag filter. The filter material 32′ of the bag filter may comprise five layers and may have a minimum pore size of 0.5 micron. The second filtrate enters the third filtration device 30′ at a pressure of around 230 kPa. The second filtrate passes through the filter material 32′ of the third filtration device 30′, thereby removing particles having a particle size of above 0.5 micron to obtain a third filtrate.

The third filtrate is then fed into the ultrafiltration device 40′ to obtain an ultrafiltration retentate. Optionally, diafiltration using pure water or a buffer solution can be performed during ultrafiltration in accordance with known methods.

The ultrafiltration retentate is then fed into the drying device 60′ which may be in the form of a spray dryer. The ultrafiltration retentate is sprayed into a stream of hot air to obtain a powder having greater than 70 % by weight native potato protein.

The following advantages of the present disclosure are apparent.

Significantly less fouling of the filter membranes is apparent when compared to prior art methods. The applicant estimates that approximately 25× more protein passes through the filter membranes compared to prior art methods, without much process downtime. This can result in an increase in the flow rate of potato fruit juice through the apparatus 1, 1′, meaning that protein can be produced at a faster rate when compared to prior art methods. In particular, by having three filtration stages prior to the ultrafiltration stage, protein fouling can be reduced. Therefore, the apparatus 1, 1′ of the present disclosure can enable a reliable production rate to be achieved with minimal interruption.

The native potato protein obtained as described herein is a functional, clean label protein. In other words, the protein contains many of the natural biologically active components needed to support and maintain a healthy immune system.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the examples described herein may be combined with one or more features from any other example(s) described herein.

Protection may be sought for any features disclosed in any one or more published documents referenced herein in combination with the present disclosure.

Although certain example examples of the invention have been described, the scope of the appended claims is not intended to be limited solely to these examples. The claims are to be construed literally, purposively, and/or to encompass equivalents.

1. Apparatus for extracting protein from potatoes, the apparatus comprising:
a first filtration device having a filter material with a first pore size;
a second filtration device having a filter material with a second pore size;
a third filtration device having a filter material with a third pore size; and
an ultrafiltration device,
wherein the second pore size is smaller than the first pore size, and the third pore size is smaller than the second pore size.

2. The apparatus according to claim 1 further comprising:
any one or both of a decanter centrifuge and a sedimentation vessel.

3. The apparatus according to claim 1, wherein:
the first pore size is from 10 micron to 200 micron.

4. The apparatus according to claim 1, wherein:
the second pore size is from 0.8 micron to 10 micron.

5. The apparatus according to claim 1, wherein:
the third pore size is less than 0.8 micron, and optionally wherein the third pore size is 0.1 micron or above.

6. (canceled)

7. The apparatus according to claim 1, wherein:
the ultrafiltration device comprises a filter membrane having a molecular weight cut-off of from 5 kDa to 150 kDa.

8. The apparatus according to claim 1, wherein:
the filter material of any one or more of the first filtration device, the second filtration device and the third filtration device is in the form of a bag filter.

9. The apparatus according to claim 1 wherein:
the first filtration device comprises or consists of a centrifugal separator comprising:
a housing having:
an inlet for receiving potato fruit juice;
a first outlet; and
a second outlet;
an element configured to rotate relative to the housing to create centrifugal forces within the housing; and
the filter material of the first filtration device, wherein the first outlet is configured to receive retentate that has been retained by the filter material and the second outlet is configured to receive filtrate that has passed through the filter material, and optionally wherein the filter material of the second filtration device and/or the filter material of the third filtration device is in the form of a bag filter.

10. (canceled)

11. The apparatus according to claim 1, wherein:
the filter material of any one or more of the first filtration device, the second filtration device and the third filtration device comprises one or more layers.

12. The apparatus according to claim 9, wherein:
the filter material of the first filtration device and/or the filter material of the second filtration device comprises one layer.

13. The apparatus according to claim 9, wherein:
the filter material of the third filtration device comprises two or more layers.

14. The apparatus according to claim 9, wherein:
when the filter material of any one of the first filtration device, the second filtration device and the third filtration device comprises two or more layers, the layers have differing pore sizes, and optionally wherein the filter material of any one of the first filtration device, the second filtration device and the third filtration device comprises a first side and a second side opposite to the first side, wherein the layers have diminishing pore sizes going from the first side to the second side.

15. (canceled)

16. The apparatus according to claim 1, further comprising:
a drying device for drying potato protein.

17. A method for extracting protein from potatoes, the method comprising:
(a) obtaining potato fruit juice;
(b) passing the potato fruit juice through a first filtration device having a filter material with a first pore size to obtain a first filtrate;
(c) passing the first filtrate through a second filtration device having a filter material with a second pore size to obtain a second filtrate;
(d) passing the second filtrate through a third filtration device having a filter material with a third pore size to obtain a third filtrate; and
(e) ultrafiltering the third filtrate to obtain an ultrafiltration retentate, wherein the second pore size is smaller than the first pore size, and the third pore size is smaller than the second pore size.

18. The method according to claim 17, further comprising:
drying the ultrafiltration retentate to produce the potato protein.

19. The method according to claim 17, wherein:
the first pore size is from 10 to 200 micron.

20. The method according to claim 14, wherein:
the second pore size is from 0.8 micron to 10 micron.

21. The method according to claim 17, wherein:
the third pore size is less than 0.8 micron, optionally, wherein the third pore size is 0.1 micron or above.

22. (canceled)

23. The method according to claim 17, wherein:
the potato fruit juice is clarified prior to passing through the first filtration device, and optionally wherein the potato fruit juice is clarified by using a decanter centrifuge or sedimentation.

24 (canceled)

25. The method according to claim 17, wherein:
step (e) involves subjecting the ultrafiltration retentate to diafiltration.
